(19) United States
(12) Patent Application Publication
(10) Pub. No.: US 2024/0293442 A1
(43) Pub. Date: Sep. 5, 2024

(54) ASSOCIATION OF MOLECULAR OXYGEN AND HYALURONIC ACID OR ITS SALTS FOR USE IN THE TREATMENT OF SYMPTOMATOLOGY ASSOCIATED WITH VULVAR VESTIBULITIS

(71) Applicant: Federico Montanari, Argelato (BO) (IT)

(72) Inventor: Federico Montanari, Argelato (BO) (IT)

(21) Appl. No.: 18/572,550

(22) PCT Filed: Jun. 21, 2022
(86) PCT No.: PCT/IB2022/055754

§ 371 Date: Dec. 20, 2023

(30) Foreign Application Priority Data
Jun. 24, 2021 (IT) 102021000016598

(51) Int. Cl. A61K 31/728; A61K 9/00; A61K 33/00; A61M 25/00; A61P 15/02; A61P 29/00

(52) U.S. Cl. CPC A61K 31/728; A61K 9/0034; A61K 33/00; A61P 15/02; A61P 29/00; A61M 25/007

(57) ABSTRACT

The object of the invention relates to an association of molecular oxygen and hyaluronic acid or its salts for use in the treatment of symptomatology associated with vulvar vestibulitis.

### FIELD OF THE INVENTION

The field of the present invention relates to an association of molecular oxygen and hyaluronic acid or its salts for use in the treatment of symptomatology associated with vulvar vestibulitis.

### BACKGROUND ART

Vulvar Vestibulitis (VV) is a chronic, heterogeneous, multifactorial and multi-systemic disease (Graziottin A, Brotto L A. Vulvar vestibulitis syndrome: a clinical approach. J Sex Marital Ther. 2004 May-June; 30(3): 125-39). Friederich first described it in 1987 as a disease characterized by three particular symptoms and signs which are: significant pain on contact with the vaginal vestibule and dyspareunia, fragility of the vestibular tissue, detectable on contact, and objective evidence of vestibular erythema of varying degrees (Friedrich, E. G. (1987). Vulvar Vestibulitis Syndrome. Journal of Reproductive Medicine, 32, 110-4).

It is the main cause of dyspareunia in women of childbearing age (Graziottin A. Etiology and diagnosis of coital pain. J Endocrinol Invest. 2003; 26(3 Suppl): 115-21; Bergeron S, Binik Y M, Khalifé S, Pagidas K. Vulvar vestibulitis syndrome: a critical review. Clin J Pain. 1997 March; 13(1): 27-42) and affects 8-12% of women who visit a gynaecological clinic (Goetsch M F. Vulvar vestibulitis: prevalence and historic features in a general gynecologic practice population. Am J Obstet Gynecol. 1991 June; 164(6 Pt 1): 1609-14; discussion 1614-6).

As a multi-systemic disease, VV has a complex pathophysiology. Bornstein demonstrated that the mucosal structure of the vestibule is susceptible to a mast cell mediated inflammatory response (Bornstein J, Goldschmid N, Sabo E. Hyperinnervation and mast cell activation may be used as histopathologic diagnostic criteria for vulvar vestibulitis. Gynecol Obstet Invest. 2004; 58(3):171-8). In fact, in the population affected by VV, a significantly higher number of mast cells in the superficial layers of the vestibular mucosa is found with respect to the deep ones and, at the same time, a significant increase in the production, deposition and release of multiple mediators of inflammation. Hence, chronic inflammation could be responsible for the thinning and brittleness of the typical introital mucosa of VV.

Mast cell up-regulation increases the production of cytokines, substance P, and other inflammatory mediators which cause vasodilation, exudation, oedema, swelling, redness, and pain. The production of Nerve Growth Factor (NGF) by the hyperactive mast cell stimulates the proliferation of pain nerve fibres; extending to the most superficial layers of the mucosa, these cause both an increase in the perception of pain (hyperalgesia) and a perceptual shift from a tactile type of pain to one characterized by burning (allodynia) (Bohm-Starke N, Hilliges M, Falconer C, Rylander E. Increased intraepithelial innervation in women with vulvar vestibulitis syndrome. Gynecol Obstet Invest. 1998; 46(4): 256-60).

The activation of mast cell degranulation is mediated in different modes and times by different agonist stimuli which contribute to the heterogeneity of the clinical presentation. Infections, mechanical trauma from rubbing during sexual intercourse, if the lubrication is insufficient, oestrogen, physical-chemical stimuli and allergens can all activate the release of mast cell mediators. These can promote inflammation by showing the typical clinical features (rubor, tumor, calor, dolor et functio laesa, the latter understood as an impossibility of sexual intercourse).

The first symptom of VV is represented by acute pain (nociceptive), caused by ongoing inflammation and injury to which the body seeks to react and adapt (Bohm-Starke N, Hilliges M, Falconer C, Rylander E. Increased intraepithelial innervation in women with vulvar vestibulitis syndrome. Gynecol Obstet Invest. 1998; 46(4):256-60). As time passes, the pain can become chronic and become disease itself (neuropathic), being generated by the nerves themselves or by the higher centres. The up-regulation of the pain system stimulates the adrenergic one, inducing the activation of the neurovegetative system responsible for the painful hyperaesthesia, the possible defensive posture and modifications of the pain threshold (Granot M, Friedman M, Yarnitsky D, Zimmer E Z. Enhancement of the perception of systemic pain in women with vulvar vestibulitis. BJOG. 2002 August; 109(8):863-6).

The pain often provokes a defensive contraction of the muscles of the affected area, so as to minimize further injury (Weiss J M. Pelvic floor myofascial trigger points: manual therapy for interstitial cystitis and the urgency-frequency syndrome. Urol. 2001 December; 166(6):2226-31). The contraction of the levator ani can moreover be pre-existent to the VV, if associated with vaginism as predisposing factor, or acquired in response to a persistent introital pain (Graziottin A. Why Deny Dyspareunia Its Sexual Meaning? Archives of Sexual Behaviour, 34, 1, 32-34, 57-61, author reply 63-67, 2005).

Recent research suggests that the difficulty in inserting internal swabs in adolescents could be the symptom-indicator of a hyperactivity of the levator ani muscle; in fact, an excessive contraction of the latter is such as to (reversibly) restrict vaginal entry. This hypertone would become a predisposing factor both to make penetration persistently painful from the beginning of the sexual life (“lifelong”, or primary, dyspareunia), and to cause micro-trauma of the vestibule in the case of sexual intercourse, thus precipitating the VV.

Regarding the involvement of the hormonal system, VV is a typical disorder of childbearing age. In some women, premenstrual flares are typical. A hypersensitivity of the mast cell to oestrogens has been hypothesized. In fact, oestrogens are agonists of mast cell degranulation. After menopause, recurrences of VV have been observed during vaginal and/or systemic hormone replacement therapy, in synergy with recurrent candidiasis. This is due to the interaction between oestrogens and vulnerability to candida infections, and between oestrogens and mast cell degranulation.

The vascular system is activated during chronic inflammation. Vestibular erythema is the epiphenomenon of superficial vasodilation mediated by the calcitonin gene peptide (CGRP) (Bohm-Starke N, Hilliges M, Falconer C, Rylander E. Neurochemical characterization of the vestibular nerves in women with vulvar vestibulitis syndrome. Gynecol Obstet Invest. 1999; 48(4): 270-5) released by the C nociceptors which can cause vasodilation and an activation of the axonal reflex even at low activity levels. Neurogenic inflammation describes the acute vasodilatation mediated by the nerve signals which move in retrograde along the sensory nerves and could activate both degranulation and vasodilatation.

VV should be suspected in the following cases: when a woman complains of superficial dyspareunia, with burning and/or acute pain, upon introital contact, or when upon clinical examination, the three signs described by Friederich are found. Instrumental examinations are currently used mainly in research.

Medical history is carried out on several fronts: general medical evaluation, sexological history, perceptions of symptoms.

As regards the general medical evaluation, in addition to the traditional medical history, the following are also recorded: medical pelvic co-morbidities (urological disorders of the lower urinary tract, recurrent cystitis, interstitial cystitis, urinary urgency, enuresis); iatrogenic vaginal pain (outcomes of episodiorrhaphy, colpoplastic, especially if posterior, radical vaginal surgeries and/or pelvic radiotherapies); constipation, fissures, haemorrhoids; systemic co-morbidities such as diabetes, as it favours recurrent candida infections; infections, also in other organs, requiring repeated antibiotic therapies, which often result in recurrent candida infections; allergies and atopies; depressions (Graziottin A, Brotto L A. Vulvar vestibulitis syndrome: a clinical approach. J Sex Marital Ther. 2004 May-June; 30(3): 125-39); previous or ongoing vaginal infections, or sexually transmitted diseases such as candida, gardnerella, HPV and genital herpes, which deserve specific attention by the doctor, as predisposing or precipitating factors to VV (Graziottin A. Nicolosi A. E. Caliari I. Vulvar Vestibulitis and dyspareunia: pain map and medical diagnosis Abstract Book, Annual Meeting Female Sexual Function Forum, Boston, USA, Oct. 25-28, 2001, p. 160). If affirmative, the vaginal environment should then be evaluated with a vaginal swab and a cultural examination, especially with suspected infectious; finally, all pharmacological treatments, ongoing or previous, aimed at treating both other conditions and VV, must be taken into account. Any ongoing hormonal treatment (oral contraceptive therapy, hormone replacement therapy, etc.), and all systemic and local treatments should therefore be documented.

The sexological medical history is focused on any dysfunctions prior to the appearance of VV. The following are recorded: the sexual practices of a woman, possible coexistence with any sexual dysfunction, primary (“lifelong”) or acquired (secondary), possible dysfunction of desire, arousal, orgasm and dyspareunia. In young women, the possible symptoms associated with VV deserve particular attention, including the difficulty of using vaginal swabs and the fear of penetration, as well as any post-coital urinary symptoms (cystalgia and/or post-coital urethralgia, dysuria, urinary urgency).

Finally, the perception that women have of the duration and characteristics of symptoms is assessed, i.e., how the disease is experienced both as physical suffering and as an interaction with emotional, work and social activities.

The physical examination can document the presence of general defensive postures (i.e., characterized by excessive increase in general muscle tone and refusal of any genital contact, including objective vaginal examination) and primary dyspareunia. These signs and symptoms could be associated with primary vaginism concomitant with VV (Graziottin, A. (2001). Clinical approach to dyspareunia. Journal of Sex and Marital Therapy, 27, 489-501). In addition, the examination evaluates the vaginal pH through a vaginal stick during the gynaecological examination since it must always be recorded in the file (Graziottin A, Brotto L A. Vulvar vestibulitis syndrome: a clinical approach. J Sex Marital Ther. 2004 May-June; 30(3): 125-39). Finally, the examination also allows the quality of the pain to be evaluated. In fact, three basic questions should be asked during the gynaecological examination: “Where does it hurt?”, “When does it hurt?”, and “What symptoms accompany the pain?”. These three questions are essential since the site and characteristics of pain onset are the most robust predictors of the biological etiology of pain itself (Meana M, Binik Y M, Khalife S, Cohen D. Dyspareunia: sexual dysfunction or pain syndrome? J Nerv Ment Dis. 1997 September; 185(9):561-9) and should introduce the next step regarding the objectification of the clinical data. Pain mapping is another relevant fact; the precise identification of tenderness indicates the external, middle third and distal vaginal genitalia. The precise location of the pain, its beginning, its characteristics are the main predictive signs of its organic nature. Finally, the examination also allows a quantification (score) of the pain. This involves bilateral quantification of pain intensity perception using Likert's visual analog scale (from 0: no pain, to 10: more intense pain).

### Problems of the Prior Art

The treatment of vulvar vestibulitis can include several approaches. For example, pain modulation is possible by applying topical drugs such as lidocaine (local anaesthetic) or chromoglycated sodium to the vaginal vestibule, which stabilizes the membranes of white blood cells, including those of mast cells, interrupting the neurogenic inflammation underlying the problem. The pharmacological therapy can also include the use of specific analgesics, such as amiptriptyline or gabapentin. Sometimes, systemic drugs such as tricyclic antidepressants, anticonvulsants, are indicated to interrupt the circuits of chronic pain.

Women with hypertone of the vaginal muscles can benefit from physiotherapy (pelvic floor training) and from the electromyographic biofeedback of the pelvic muscles (self-relaxation technique which teaches how to better control the contractions of the muscles and the pain they cause).

In some extreme cases, surgery has been performed to remove the proliferated nerve endings of the proximal margin of the lower vagina and the innermost part of the small lips (hymen excision).

Therefore, a need remains for treating the symptomatology associated with vulvar vestibulitis with the use of active associations with reduced or no side effects as opposed to conventional drug therapy.

### SUMMARY OF THE INVENTION

A first object of the present invention is an association comprising molecular oxygen and hyaluronic acid or its salts for use in the treatment of symptomatology associated with vulvar vestibulitis.

A further object of the present invention is a kit for use in the treatment of symptomatology associated with vulvar vestibulitis.

### Advantages of the Invention

The association is particularly advantageous since it combines the effects of molecular oxygen and hyaluronic acid at the genital tissue level, preferably at the external genital tissue level, in an enhanced and/or synergistic manner. This means that the oxygen and hyaluronic acid comprised in the association according to the invention support, promote and assist each other's action. Thereby, it is possible to achieve an improvement in the symptomatology associated with vulvar vestibulitis and, consequently, an improvement in the quality of life of the subjects affected by this disease. The Applicant has also demonstrated its efficacy in the experimental test of Example 1.

Oxygen therapy has a powerful regenerative, antibacterial and biostimulant effect; in this sense, the Applicant believes it can also be useful in the treatment of VV, specifically for use in the treatment of the symptomatology associated therewith, such as, by way of example, redness, burning, pain, recurrent infections, dyspareunia, pruritis.

It is known that oxygen therapy increases the availability of oxygen to the tissues, promotes the increase of tissue repair processes and increases collagen synthesis, allowing a normal hydroxylation of this protein. In fact, at tissue oxygen tensions below normal, collagen is not correctly synthesized, slowing the healing of ulcers and wounds (Hunt T K, Pai M P. The effect of varying ambient oxygen tensions on wound metabolism and collagen synthesis. Surg Gynecol Obstet. 1972; 135(4):561-567). In addition, oxygen induces a neo-angiogenetic stimulus by releasing factors such as Vascular Endothelial Growth Factor (VEGF) (Sheikh A Y, Gibson J J, Rollins M D, et al. Effect of hyperoxia on vascular endothelial growth factor levels in a wound model. Arch. Surg. 2000; 135:1293-1297). This function is essential for the restoration of microcirculation in compromised vascular situations, restoring a vascular flow in hypoxic areas which ensures proper tissue regeneration (Tandara, A. & Mustoe, T. Oxygen in Wound Healing-More than a Nutrient. World J. Surg. (2004) 28: 294).

Hyaluronic acid is a natural polysaccharide which is a fundamental part of the extracellular matrix of the skin and cartilage. Hyaluronic acid exhibits remarkable adhesive, moisturising and repairing properties of the vaginal mucosa (Chen J et al. “Evaluation of the efficacy and safety of hyaluronic acid vaginal . . . ” J Sex Med. 2013; 10: 1575-84).

An association of high-concentration oxygen and hyaluronic acid, for use in the topical treatment of vaginal disorders chosen from vaginal dryness and vulvovaginal atrophy (AVV), is the subject of European patent application No. 3645016.

### DESCRIPTION OF THE FIGURES

FIG. 1—Depiction of the cannula-type dispensing device;

FIG. 2—Depiction of the aerograph-type dispensing device;

FIG. 3—Table 1 showing the evaluation parameters related to the experimental test of Example 1;

FIG. 4—Graphic depiction of the results after application of the administration protocol of Example 1.

### DETAILED DESCRIPTION OF THE INVENTION

As reported in the prior art, vulvar vestibulitis is a condition considered to be a chronic pain syndrome, i.e., the pain tends to persist even for years if not relieved by treatment. It occurs whenever pressure is exerted on the area around the vaginal entrance.

For the purposes of the invention, vulvar vestibulitis is defined as an inflammation of the vestibule. The vestibule is part of the vulva, which forms the set of external female genital organs. In particular, the vulva is positioned below the mons pubis (the skin area above the pubic symphysis), and consists of the labia majora and minora, the clitoris, the vulvar vestibule, the urethral orifice and the vaginal orifice.

Preferably, the symptomatology associated with vulvar vestibulitis comprises at least recurrent infections and/or increased muscle tone. Preferably, the symptomatology associated with vulvar vestibulitis comprises at least one of the further symptoms selected from: fragility of the vestibular tissue to touch and vulvar lesions.

The symptomatology associated with vulvar vestibulitis is preferably selected from the group consisting of: redness, preferably of the mucosa of the vulva; burning, preferably at the vaginal entrance (at 5 and 7 o'clock, if the vaginal entrance is imagined as the dial of a clock); pain; preferably acute and/or chronic pain; recurrent infections; dyspareunia; pruritus; fragility of the vestibular tissue to the touch; thinning and brittleness of the introitus mucosa; lesions; preferably vulvar lesions; increased muscle tone and mixtures of the foregoing.

### Association

As anticipated, the association comprises molecular oxygen (or gaseous oxygen) and hyaluronic acid or its salts. According to a preferred embodiment, the association consists of molecular (or gaseous) oxygen and hyaluronic acid or its salts.

The association comprises molecular oxygen (or gaseous oxygen) with a degree of purity of ≥90% (v/v), preferably comprised between 90% and 99%, preferably comprised between 90% and 96% (v/v), preferably comprised between 92% and 95% (v/v), preferably equal to 95% (v/v).

The molecular oxygen (or gaseous oxygen) comprised in the association can be obtained using methodologies known to those skilled in the art; preferably, the molecular oxygen is obtained thanks to an oxygen concentrator, which draws external air and concentrates it in the oxygen fraction. The maximum dispensing pressure by means of the oxygen concentrator is comprised preferably between 50 and 150 kPa, preferably it is 100 kPa.

The molecular oxygen dosage to be administered is preferably comprised between 5 and 130 L, preferably comprised between 5 and 120 L, preferably between 10 and 130 L, preferably comprised between 10 and 120 L, preferably between 20 and 120 L, preferably between 10 and 90 L, preferably between 10 and 70 L.

According to a preferred form of the invention, the molecular oxygen is dispensed at a flow comprised preferably between 0.5 and 7 L/min, preferably between 1 and 6 L/min, preferably between 1.5 and 4.5 L/min, preferably equal to 3 L/min.

The association preferably comprises low molecular weight hyaluronic acid; preferably, the association comprises hyaluronic acid having a molecular weight comprised between 200 kDa and 2 MDa.

According to a preferred embodiment, the hyaluronic acid is preferably dissolved in an aqueous solution at a concentration preferably comprised between 0.050% and 0.50% by weight, preferably comprised between 0.050% and 0.30% by weight, preferably comprised between 0.10% and 0.25% by weight, preferably equal to 0.20% by weight on the total volume of the aqueous solution.

Preferably, the dosage of hyaluronic acid is comprised between 10 mg and 120 mg per dosage unit. Preferably, the dosage unit contains or consists of the aqueous solution of hyaluronic acid in a volume comprised between 5 and 15 ml, preferably equal to 10 ml.

According to a preferred embodiment, the hyaluronic acid solution is to be comprised in a closed container, preferably an ampoule, bottle, vial.

Preferably, the hyaluronic acid is in the form of a hyaluronate salt, preferably sodium hyaluronate.

According to a preferred embodiment, the association is preferably applied topically. Preferably, the association is dispensed directly to the external (or vulvar) genital tissue and/or internally to the vaginal cavity. It should be noted that the association is dispensed in the form of steam (i.e., vaporized or nebulized).

The association can be dispensed in a time comprised between 5 and 20 minutes (extremes included), preferably comprised between 5 and 10 minutes, preferably equal to 15 minutes, preferably equal to 10 minutes, preferably equal to 5 minutes.

Preferably, the association is intended for the treatment of symptoms associated with vulvar vestibulitis, preferably in women of childbearing age. A woman of childbearing age is intended as a woman comprised between 18 and 40 years of age, preferably between 20 and 37 years of age.

### Administration Protocol

According to a preferred form, the association is dispensed following an administration protocol comprising the following steps:
i) dispensing the association on the external or vulvar tissue,
ii) dispensing the molecular oxygen inside the female genital apparatus or the internal vaginal cavity, and
iii) dispensing the association inside the female genital apparatus or the internal vaginal cavity, the association being that for use which is the object of the invention.

The organs and the anatomical structures situated inside the female genital apparatus are preferably: the vagina, the cervix, the uterus, the fallopian tubes and the ovaries; the organs and the anatomical structures situated outside, on the other hand, are preferably: the mons pubis, the labia majora, the labia minora, the Bartholin's glands and the clitoris.

The internal application step iii) can optionally be followed by a step of applying a manual pelvic decontracting treatment iv), preferably of physiokinesitherapy. For the purposes of the invention, manual treatment means a physiotherapeutic treatment aimed at motor rehabilitation by means of manual intervention or therapeutic exercise directed by a specialist in the field. An example of manual physiotherapy treatment is physiokinesitherapy aimed at reducing pelvic muscle contractile tone with decontracting manipulations.

For the dispensing of the association at the genital tissue level, external or internal, two types of dispensing devices can preferably be used: a cannula or an aerograph.

The cannula is preferably intended for the intravaginal dispensing of the oxygen, the hyaluronic acid or its salts and/or the association of the invention; the aerograph is preferably used for the external dispensing of the molecular (or gaseous) oxygen, the hyaluronic acid or its salts and/or the association of the invention.

The external application step of the association i) preferably has a duration comprised between 2 and 8 minutes, preferably equal to 5 minutes; the internal application step of molecular oxygen ii) preferably has a duration comprised between 7 and 13 minutes, preferably equal to 10 minutes; finally, the internal application step of the association iii) has a duration comprised preferably between 2 and 8 minutes, preferably equal to 5 minutes.

Preferably, the association is to be administered on a weekly basis, preferably once a week; in particular, the association is to be administered for a time period comprised between 5 and 40 consecutive days, preferably comprised between 5 and 10 days, preferably equal to 7 days.

### Kit

The invention further relates to a kit for use in the treatment of symptomatology associated with vulvar vestibulitis, comprising:
hyaluronic acid or its salts,
molecular oxygen;
instructions for the preparation of an association comprising molecular oxygen and hyaluronic acid or its salts;
one or more dispensing devices suitable for nebulising an association comprising molecular oxygen and hyaluronic acid or its salts,
the association being that of the invention described above.

According to a preferred embodiment, the kit also comprises an oxygen concentrator. It should be noted that the molecular oxygen included in the kit can be for example in the form of pressurized, refillable or reloadable cylinders. When the kit comprises the oxygen concentrator, the molecular oxygen is generated extemporaneously, without the need for storage.

According to a further preferred embodiment, the kit further comprises a molecular oxygen transport cable.

The hyaluronic acid or its salts is preferably comprised inside a closed container, such as an ampoule, bottle, vial. The closed container constitutes a dosage unit preferably having a volume comprised between 5 and 15 ml, preferably equal to 10 ml; according to the latter embodiment, the number of closed containers comprising hyaluronic acid or its salts is equal to both the number of cannulas and the number of molecular oxygen transport cables.

Hyaluronic acid salt is preferably understood as sodium hyaluronate.

The hyaluronic acid or its salts is preferably in the form of an aqueous solution of hyaluronic acid, preferably in a concentration comprised between 0.050% and 0.30% by weight, preferably comprised between 0.10% and 0.25% by weight, preferably equal to 0.20% by weight on the total volume of the aqueous solution. According to a preferred embodiment, the nebulisation of the association comprising molecular oxygen and hyaluronic acid is possible directly on the areas affected by the lesions.

According to a preferred embodiment, the kit comprises a disposable dispensing device, preferably a vaginal cannula. It should be noted that the vaginal cannula constitutes a vaginal dispensing (or applicator) device.

A disposable dispensing device is intended as a device which is used for a single time or single treatment/protocol, then disposed of, i.e., not reusable a second time.

With reference to FIG. 1, we describe below the cannula device 100, configured for dispensing the association according to the invention.

The cannula 100 comprises a hollow container body 100a, extending between two ends, along a horizontal axis (X-X): a proximal end 101 and a distal end 102. The proximal end 101 comprises an opening (not shown in FIG. 1), arranged on a flat surface in section with respect to the container body of the cannula. The opening comprises anchoring means for connecting the proximal end 101 to a molecular oxygen transport cable of an oxygen concentrator.

The container body 100a comprises an internal cavity, intended to receive the molecular oxygen flow coming from the molecular oxygen transport cable and possibly an aqueous solution of hyaluronic acid; and a hole 100b which crosses the container body 100a putting the internal cavity in fluid communication with the outside.

The distal end 102 comprises a head portion 102a, of ovoid or rounded shape, provided with at least two holes 102b for nebulising the association of molecular oxygen and hyaluronic acid or its salts. Preferably, the head portion 102a is in a single piece with the container body 100a.

It should be noted that the head portion 102a is also hollow and in flow communication with the hollow container body 100a of the cannula.

According to a preferred embodiment, the head portion 102a comprises at least two shaped ribs 102c, which protrude from the outer surface of the head portion 102a, defining depressions 102d where the at least two holes 102b are located. The holes 102b cross the head portion 102a, putting the hollow container body 100a of the cannula in fluid communication with the exterior.

Preferably, there are four shaped ribs 102c which are arranged crosswise on the head portion 102a. The ribs are preferably tapered, widening towards the distal end 102 and narrowing towards the proximal end 101, so as to give the head portion 102a a comfortable shape for intravaginal insertion.

According to a preferred embodiment, the holes 102b are located in pairs (in number equal to 2) on each of the depressions defined by the ribs 102c.

It should be noted that, advantageously, the ribs 102c thus shaped allow, upon use, to remove the mucosal tissues from the outer surface of the cannula, facilitating the exit of the molecular oxygen and/or the association comprising molecular oxygen and hyaluronic acid.

It should be noted that the molecular oxygen transport cable is the cable of an oxygen concentrator. Preferably, the molecular oxygen transport cable is of a material suitable for transporting molecular oxygen (or gaseous oxygen), preferably it is in polymeric material, preferably it is in polypropylene PP. The oxygen concentrator is an instrument known in the background art and commercially available, configured to draw in outside air and filter it, to obtain air with an oxygen titre ≥80%.

It should be noted that a generic oxygen concentrator preferably comprises:
a compressed air generator or compressor configured to draw air from the external environment, filter and compress it.
a machine body, comprising or provided with molecular zeolite sieves. The machine body uses the principle of different absorption of gas molecules to allow O₂ to pass through and to retain the other gases in the air, such as nitrogen, argon, helium and hydrogen. The machine body transforms the outside air into 93-95% pure oxygen.
oxygen transport cable, provided with anchoring means suitable for engaging a dispensing device, said dispensing device preferably being chosen between vaginal cannula and aerograph.

According to a further embodiment, the kit for use according to the invention comprises a multi-use (or reusable) dispensing device, preferably an aerograph.

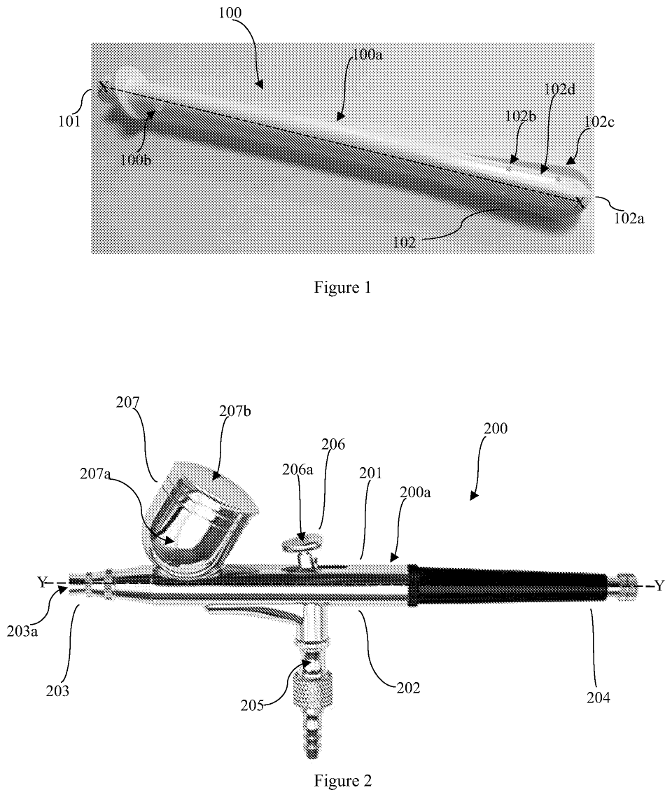

Preferably, the kit comprises both a disposable dispensing device, preferably a vaginal cannula, and a multi-use (or reusable) dispensing device, preferably an aerograph. With reference to FIG. 2, a preferred embodiment of an aerograph suitable for the purposes of the invention is described below.

The aerograph 200 comprises a hollow container body 200a, having an upper surface 201 and a lower surface 202; the hollow container body extends between two ends, along a horizontal axis Y-Y: a proximal end 203 and a distal end 204.

The container body comprises an internal cavity, suitable for receiving a pressurized molecular oxygen flow.

The distal end 204 consists of a handle for use by a user.

The proximal end 203 comprises an opening (not shown in the figure), arranged on a flat surface in section with respect to the container body 200a of the aerograph, which puts the internal cavity in fluid communication with the outside. The opening comprises a nozzle 203a for the exit of molecular oxygen or the association of molecular oxygen and hyaluronic acid.

The aerograph comprises a duct 205, in fluid communication with the internal cavity of the container body, arranged between the proximal end 203 and the distal end 204, and protruding from one of the surfaces of the container body 200a.

The duct 205 comprises anchoring means for the connection to a molecular oxygen transport cable of an oxygen concentrator.

The container body 200a further comprises a valve assembly 206 for adjusting the molecular oxygen flow passing in the internal cavity of the container body. The valve assembly 206 comprises a lever or button 206a which adjusts the valve position to modulate the oxygen flow. The lever or button is preferably located on the upper surface 201 of the aerograph, between the distal end 204 and the proximal end 203.

The aerograph comprises a tank 207, arranged on the upper surface 201 of the container body 200a, the tank 207 comprising an internal concavity 207a and a lid 207b. The internal concavity 207a comprises an opening in fluid communication with the internal cavity of the container body; such an internal concavity 207a, suitable for receiving a liquid, allows said liquid to flow out into the internal cavity of the container body.

Upon use, when the duct 205 is anchored to the molecular oxygen transport cable of a pressure generator and the pressure generator is operated, the outflow of liquid from the tank 207 to the container body 200a of the aerograph is induced by the formation of a pressure difference between the internal cavity of the container body and the internal concavity 207a of the tank.

The aerograph 200 is preferably made of material suitable for withstanding sterilizations (such as autoclaving or the like), preferably it is made of steel.

The aerograph is preferably used for the dispensing/nebulisation at the level of the external genitalia. In other words, the aerograph constitutes a vulvar dispensing device (or applicator).

### Examples

The Applicant gives examples below merely for illustrative and non-limiting purposes of the invention.

### Example 1—Experimental Test

### 1.1 Materials and Methods

Five weekly oxygen therapy treatments were performed on 25 women diagnosed with vestibulitis, for a total of five weeks.

The administration protocol comprises the following steps:
i) dispensing of oxygen and nebulized hyaluronic acid with a vulvar applicator for a period of five minutes (external application step of the association),
ii) subsequently vaginal oxygen was applied for 10 minutes (internal application step of molecular oxygen (or gaseous oxygen)) and
iii) dispensing of oxygen and hyaluronic acid for an additional 5 minutes (internal application step).

For the treatment, a gynaecological oxygen therapy device was used which allows the topical administration of oxygen with a high degree of purity of up to 93±3%, at a flow comprised between 1 and 6 L/min (litres/minute).

All the patients have a hypertone of the pelvic muscles: in addition to the treatment of vulvar and vaginal oxygen therapy, a manual treatment of physiokinesitherapy (step of application of a manual treatment iv)) was applied, after the dispensing of oxygen and hyaluronic acid, with the aim of treating the pelvic contracture. It should be noted that, according to the Applicants, such a treatment is optional and, based on current data, irrelevant for the purposes of the final therapeutic outcome.

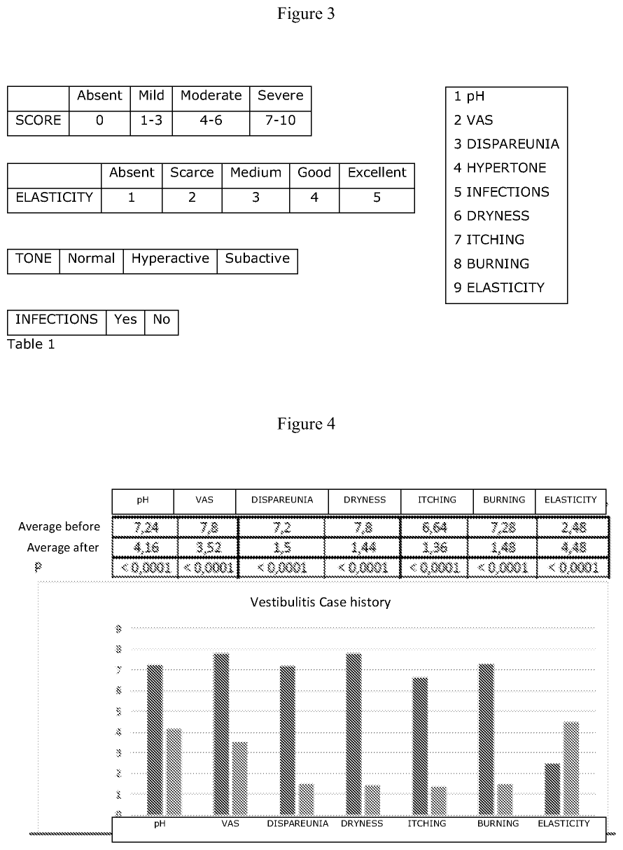

A double evaluation was carried out on the treated subjects: a subjective evaluation of the patients and one by the doctor. The subjective scale was compiled by means of an analogue scale which evaluated the pain during sexual intercourse (dyspareunia), dryness, itching and burning with a VAS scale from 0 to 10, where 10 represents the maximum intensity and 0 the absence of the disorder, analysing the symptoms before the first treatment session (T0) and at the end of the 5 sessions (T5). The doctor instead evaluated parameters such as pH, elasticity, tone and the presence or absence of infections (Table 1, FIG. 3).

### 1.2 Results

Patients reported a significant improvement of all the indexes analysed as shown in FIG. 4, with the greatest effect regarding pain reduction (VAS T0=7.2; VAS T5=1.5, Wilcoxon signed-rank test P<0.0001), but also regarding burning (VAS T0=7.28; VAS T5=1.48, Wilcoxon signed-rank test P<0.0001) and itching (VAS T0=6.64; VAS T5=1.36, Wilcoxon signed-rank test P<0.0001).

Regarding the elasticity index, the variation between T0 and T5 appears highly significant (Wilcoxon signed-rank test P<0.0001) with an increase in the index from 2.48 to 4.48.

The data regarding the effect of the treatment on tone show that in all cases there was an improvement in symptomatology (Table 2-Comparison between the condition of muscle tone and the presence of infections of various etiologies before and after the application of the administration protocol of Example 1).

| | TONE | | INFECTIONS | |
|---|---|---|---|---|
| | BEFORE | AFTER | BEFORE | AFTER |
| 1 | Hyper | Normal | Yes | No |
| 2 | Hyper | Normal | Yes | No |
| 3 | Hyper | Normal | Yes | No |
| 4 | Hyper | Normal | Yes + cystitis | No |
| 5 | Hyper | Normal | Yes | No |
| 6 | Hyper | Normal | Yes | No |
| 7 | Hyper | Normal | Yes | No |
| 8 | Hyper | Normal | Yes | No |
| 9 | Hyper | Normal | Yes | No |
| 10 | Hyper | Normal | / | / |
| 11 | Hyper | Normal | / | / |
| 12 | Hyper | Normal | Yes | No |
| 13 | Hyper | Normal | Yes | No |
| 14 | Hyper | Normal | Yes + cystitis | No |
| 15 | Hyper | Normal | / | / |
| 16 | Hyper | Normal | / | / |
| 17 | Hyper | Normal | Yes | No |
| 18 | Hyper | Normal | Yes | No |
| 19 | Hyper | Normal | Yes | No |
| 20 | Hyper | Normal | Yes | No |
| 21 | Hyper | Normal | / | / |
| 22 | Hyper | Normal | / | / |
| 23 | Hyper | Normal | / | / |
| 24 | Hyper | Normal | Yes | No |
| 25 | Hyper | Normal | Yes | No |

Of the 25 women included in the trial, 18 had infections at T0, confirming the association between vestibulitis and vulnerability to infections. At the end of the treatment (T5), all the detected infections had resolved (Table 2 of FIG. 5), demonstrating the effectiveness of the combined treatment of high concentration oxygen and hyaluronic acid.

No side effects associable with the treatment were reported by the patients.

1-11. (canceled)

12. A method for treating the symptomatology associated with vulvar vestibulitis comprising administering topically to a subject in need of this treatment an association comprising molecular oxygen and hyaluronic acid or salts thereof.

13. The method according to claim 12, wherein the molecular oxygen has a degree of purity ≥90% (v/v).

14. The method according to claim 12, wherein the molecular oxygen is administered at a dosage ranging from 5 to 130 liters.

15. The method according to claim 12, wherein the hyaluronic acid has a molecular weight ranging from 200 kDa to 2 MDa.

16. The method according to claim 12, wherein hyaluronic acid is administered at unitary dosages comprised between 10 mg and 120 mg.

17. The method according to claim 12, wherein the hyaluronic acid is administered in the form of an aqueous solution at a concentration comprised between 0.05% and 0.5% by weight on the total volume of the aqueous solution.

18. The method according to claim 12, wherein the hyaluronic acid is in the form of sodium hyaluronate.

19. The method according to claim 12, wherein the symptomatology associated with vulvar vestibulitis is selected from the group consisting of: redness, burning, pain, recurrent infections, dyspareunia, itching, fragility of vestibular tissue to the touch, thinning and friability of the introital mucosa, lesions, increased muscle tone and mixtures thereof.

20. The method according to claim 12, wherein hyaluronic acid and oxygen is administered with a kit comprising:
hyaluronic acid or salts thereof;
molecular oxygen;
instructions for the preparation of the association comprising molecular oxygen and hyaluronic acid or salts thereof; and
one or more dispensing devices suitable for the nebulization of the association comprising molecular oxygen and hyaluronic acid or salts thereof.

21. The method according to claim 20, wherein the dispensing device is a disposable vaginal cannula (100) and/or a spray gun.

22. The method according to claim 21, wherein said vaginal cannula (100) comprises
a hollow container body (100a), extending between two ends, along a horizontal axis (X-X),
the first proximal end (101), comprising an opening arranged on a flat surface in section with respect to the cannula container body, the opening comprising anchoring means for connecting the proximal end (101) to a transport cable of molecular oxygen of an oxygen concentrator,
the second distal end (102) comprising a head portion (102a) provided with at least two holes (102b) for nebulizing the association of molecular oxygen and hyaluronic acid or salts thereof, the head portion (102a) also being hollow and in flow communication with the hollow container body (100a) of the cannula,
wherein
the container body (100a) comprises an inner cavity which, when the proximal end (101) is anchored to the molecular oxygen transport cable, allows the flow of gas from the proximal end (101) to the distal end (102), and a hole (100b) configured for injecting an aqueous solution of hyaluronic acid or salts thereof into the molecular oxygen flow, the hole (100b) putting the inner cavity of the container body (100a) in fluid communication with the outside.
